(19) United States
(12) Patent Application Publication
(10) Pub. No.: US 2005/0025807 A1
(43) Pub. Date: Feb. 3, 2005

(54) Cured porous calcium phosphate material and uses thereof

(75) Inventors: Makoto Ootsuka, Hyogo (JP); Atsuo Ito, Ibaraki-ken (JP)

Correspondence Address: SUGHRUE MION, PLLC, 2100 PENNSYLVANIA AVENUE, N.W., SUITE 800, WASHINGTON, DC, 20037, US

(73) Assignee: NATIONAL INSTITUTE OF ADVANCED INDUSTRIAL SCIENCE AND TECHNOLOGY

(21) Appl. No.: 10/810,702

(22) Filed: Mar. 29, 2004

(30) Foreign Application Priority Data
Jul. 31, 2003 (JP) 2003-283968

(51) Int. Cl. A61K 9/14; A61K 38/39; A61K 38/22; A61K 38/18

(52) U.S. Cl. 424/426; 514/21; 514/12

(57) ABSTRACT

There are provided a cured porous calcium phosphate material, an alternative living body tissue material, a tissue engineering scaffold and a drug support medium for DDS using the same. The cured material includes penetration pores with a diameter of 70 μm or more, preferably 100 μm or more, disposed in a three-dimensional network structure, and having enough porosity for the penetration of blood vessels and tissues. Drugs important for promoting bone formation and preventing infection can be added thereto, whereby the drugs are controllably released.

This application claims priority to JP Application No. 2003-283968 filed Jul. 31, 2003.

### TECHNICAL FIELD

The present invention relates to a cured porous calcium phosphate material having penetration pores formed therein. The cured porous calcium phosphate material attained by the present invention may be used as a biotissue material, a tissue engineering scaffold, and a drug support medium for a drug delivery system (DDS) that requires biological affinity.

### BACKGROUND OF THE INVENTION

Biologically active substances such as antibiotic-containing drugs, growth factors, cell adhesion factors, other proteins, phosphatides, polysaccharides and hormones are commonly utilized to prevent infection, re-build living body tissues, produce tissues and differentiate cells. A drug or active substance may be administered to a living body as-is, or supported by any biomaterial for administration. Alternatively, a drug or active substance may be supported by a material and then released in a controlled manner therefrom.

Organic biomaterialpolymers may be used to support a drug or biologically active substance. However, bone tissue is mainly made of calcium phosphate. Therefore, supporting a biologically active substance on calcium phosphate that is used as an artificial bone material is a faster and more effective way to re-build the bone tissue than supporting the biologically active substance on an organic polymer. This enables production of calcium phosphate within the tissues from the very beginning.

There are at least four methods of supporting a drug or biologically active substance on acalcium phosphate biomaterial: surface adsorption on calcium phosphate, mixing into a calcium phosphate cured material, container preservation, and retention in calcium phosphate ceramic pores.

Examples of a calcium phosphate porous material on which a drug or biologically active substance is supported include: a paste containing a bone-forming substance, a calcium component and a thickener from which a bone-forming substance is released controllably; a cured material containing a biocompatible polymer, calcium phosphates, and a drug and water-soluble compounds; drug with calcium-containing glass powder and/or glass ceramicpowder; a calcium phosphate cement containing an antibiotic or a bone forming factor; a calcium phosphate cement containing an antibiotic or a physiologically active substance or a drug; a calcium phosphate cement containing an antibiotic; a calcium phosphate cement containing cells and various physiologically active substances or a drug obtained by using an amorphous calcium phosphate precursor; and a calcium phosphate cement compounded with protein, especially collagen, and the like.

The aforementioned previously known cured calcium phosphate materials containing a drug or biologically active substance has few macropores into which tissues or blood vessels can penetrate. Almost all the pores have a diameter of 70 μm or less, have a blind alley structure and do not penetrate through the porous material, although the cured calcium phosphate material itself is porous and has a number of micropores. Thus, approach and penetration of the blood vessels into previously known cured calcium phosphate materials are limited. As a result of the limitated nutrient and oxygen supply into the cured calcium phosphate materials, penetration of tissues such as bone tissue is insufficient, and the bone tissue is onlybound to the outer area ofthe calcium phosphate cured material. In addition, air that is unable to escape, but remains within the porous material, is a factor that prevents cells, tissues, or blood vessels from penetrating.

When a porous material having non-penetration pores or closed pores in a blind alley structure is used as a cell culture scaffold, the pores fill with air that is unable to escape and cell culture liquid. Therefore, cells can not penetrate into the pores. The application of the porous material to tissue engineering or regenerative medical engineering, wherein cells are externally cultured in the porous material and then returned to a patient's internal body with the porous material to repair tissues or regenerate organs, is thus limited.

Several variable factors have been used to control drug release from calcium phosphate cured material: drug concentration, porosity of the calcium phosphate cured material, and resorption properties of the cured material. Since the calcium phosphate cured material has a pore structure, the release rate of the drug contained in the pore may be represented by the Higuchi formula (T. Higuchi, J.Pharm.Sci., 52, 1145-1149, 1963):
[Formula 1]

$$\frac{{M}_{t}}{{M}_{0}} = A \sqrt{( \frac{D ɛ}{τ} ) ( 2 {C}_{s} - ɛ {C}_{d} )}$$

where Mt is the amount of drug released at time t, M₀ is the total drug amount, A is the surface area, D is the diffusion rate constant of the drug, Cs is the solubility of the drug, Cd is the concentration of the drug, and ε is porosity. Conventionally, the drug released from the cured calcium phosphate material is controlled by the drug concentration and the porosity of the cured material in accordance with the above formula, or by the resorption rate of the cured material itself within the living body.

The rate of drug release is controlled by the diffusion within the cured material. Accordingly, drug release behavior can be explained on a theoretical basis by the size and number of closed and open pores. However, the pores of the cured calcium phosphate material are pores that naturally occur during the mixing and curing steps. It is difficult to optimize the drug release rate using the naturally occurring pores and to release the drug safely and effectively to the tissues. Nogami et al., report that a diffusion layer of phenobarbitol has a thickness of about 30 microns under agitation conditions of 200 to 300 rpm at 30° C., calculated by the Nernst-Noyes-Whitney formula (Chem.Pharm.Bull., 17, 499-509, 1969).
[Formula 2]

$$\frac{ⅆ C}{ⅆ t} = \frac{DS}{V σ} ( {C}_{s} - C )$$

where C is the concentration at time t, Cs is the solubility of the drug, D is the diffusion rate constant of the drug, V is the solution volume, S is the surface area of the release body, and a is the thickness of the diffusion layer.

The following references discuss related art:
U.S. Pat. No. 5,053,212
Japanese Unexamined Patent Application Publication No. 2001-106638
Japanese Unexamined Patent Application Publication No. 9-225020
Japanese Unexamined Patent Application Publication No. 5-253286
U.S. Pat. No. 5,149,368
U.S. Pat. No. 5,262,166
U.S. Pat. No. 6,425,949
U.S. Pat. No. 5,968,253
U.S. Pat. No. 6,139,578
U.S. Pat. No. 6,277,151
Higuchi T, J.Pharm.Sci., 52, 1145-1149,1963
Nogami T et al., Chem.Pharm.Bull., 17, 499-509, 1969

Despite the advances discussed above, until the advent of the present invention, there has been no cured porous calcium phosphate material and drug-supporting cured porous calcium phosphate material, both having a number of deliberately-produced penetration pores for receiving tissues or blood vessels and for controlling drug release.

### SUMMARY OF THE INVENTION

The present invention enables one to control drug release by providing a cured porous calcium phosphate material formed with pores having a diameter of 70 microns or more and significantly longer than the diffusion layer that fully penetrate the cured porous material. In one embodiment, the present invention provides a cured porous calcium phosphate material and a drug-supporting, cured porous calcium phosphate material that have living body affinity, can be used as a biomaterial, a tissue engineering scaffold or a drug support medium for DDS and have a number of deliberately-produced penetration pores for receiving tissues or blood vessels and for controlling a drug release. Preferably, the calcium phosphate material of the invention is low-temperature curable.

The present invention provides a cured porous calcium phosphate material produced by forming a number of penetration pores in a cured calcium phosphate material. The penetration pores are produced such that their start and end points are deliberately and ideally positioned for the greatest functionality of the material. Tissues or blood vessels penetrate into the cured material through the pores. The cured material may have a drug or other biological active substance absorbed externally, and can therefore control drug release.

It has now been discovered that a cured porous calcium phosphate material including deliberately-formed, three-dimensional penetration pores with a diameter of 70 μm to 4 mm and having a porosity of 20% to 80% addresses the shortcomings of the prior art. The porous cured material can be cured at relatively low temperature, whereby various drugs can be held and released.

The cured porous calcium phosphate material of the present invention can be used as a drug controlled release body.

The cured porous calcium phosphate material of the present invention can be used as a biomaterial.

The cured porous calcium phosphate material of the present invention can be used as a tissue engineering scaffold.

The cured porous calcium phosphate material of the present invention preferably includes penetration pores with a diameter of 70 μm or more disposed in a three-dimensional network structure. The cured porous material has enough porosity for the penetration of blood vessels and tissues. Drugs important for promoting bone formation and preventing infection can be added thereto. The drug is controllably released. The porous cured material of the invention has excellent living body affinity, and can be used as artificial bone tissue, an alternative living body tissue material, a tissue engineering scaffold, or a drug support medium for drug delivery system. Tissues or blood vessels can penetrate porous cured material of the invention. Drug release can be controlled.

Embodiments of the present invention include the following:

1. A cured porous calcium phosphate material, comprising at least one penetration pore formed therein, wherein said pore has a diameter of 70 μm to 4 mm, and wherein said material has a porosity of 20% to 80%.

2. A material according to I, comprising a plurality of penetration pores arranged in a three-dimensional network structure.

3. A material according to 1, further comprising a biocompatiblepolymer.

4. A material according to 3, wherein the biocompatible polymer is at least one organic polymer selected from the group consisting of: collagen, gelatin, chitin, chitosan and hydroxypropyl methylcellulose.

5. A material according to 1, further comprising a drug.

6. A material according to 5, wherein the drug is at least one drug selected from the group consisting of: an antirheumatic agent such as di-sodium lobenzarit, bucillamine, Acralite salazosulfapyridine, farnesyl acid predonisone; an immunosuppression agent such as methotrexate, an arthrifuge such as colchicine, sulfan pyrazone, probenecid bucolome, benzbromarone; allopurinol; an antidiabetic agent such as insulin, isoinsulin, protamine zinc isgyline, glibenclamide, tolbutamide, acetohexamide, tolazamide, glybuzole and troglitazone; a sex hormone agent such as estradiol, ethinylestradiol, estriol, mestranol, progesterone, chlormadinon acetate, and methyltestosterone; a hormone agent such as gonadorelin acetate, somatolerin acetate, tetracosactide acetate, vasopressin, glucagon and epitiostanol; a protein bone growth factor such as calcitonin, interleukin-1, interleukin-6, a bone growth factor, an insulin-like simulating factor and a fibroblast growth factor; a bone metabolic improver such as alpha calcidiol, menatetrenone, elcatonin, ipriflavone, di-sodium etidronate, sodium alendronate hydrate; a cardiac such as digoxin, aminophylline, dopamine hydrochloride and milrinone; an antiarrhythmic agent such as disopyramide phosphate and pimenol hydrochloride; an antibacterial agent such as cephalexin, cephalothin sodium, gentamicin antibiotic, nitrofurantoin and fosfomycin sodium; an carcinostatic such as cytarabine, mercaptopurine, fluorourasil, 6-mercaptopurine, tegafur and methotrexate; and an anti-inflammatory agent such as indomethacin.

7. A material according to 1, which is low-temperature curable.

8. A material according to 1, which is heated and cured at 100 - 1200 degrees Celsius.

9. A material according to 1, wherein the penetration pore has a cross-sectional shape that is round, oval, polygonal, or a combination thereof.

10. A method of producing a cured porous calcium phosphate material, comprising the steps of:
disposing one or more rods in a single plane,
introducing into the space adjacent said one or more rods (a) a composition comprising a calcium phosphate cured material precursor and a liquid component, or (b) a composition comprising a calcium phosphate cured material precursor, a biocompatible polymer and a liquid component, whereby the composition surrounds the one or more rods,
curing the composition, and
removing the rods.

11. A method according to 10, wherein a plurality of rods are disposed in a single plane.

12. A method according to 11 wherein a plurality of second rods are disposed in a single plane in one or more directions on the first rods.

13. A method according to 12, wherein the second rods are disposed in different directions than the first rods.

14. A method according to 10, wherein the composition comprises a drug.

15. A method according to 10, wherein the volume percentage of the one or more rods is 5% to 90% of the cured material.

16. A method according to 10, wherein the one or more rods have a cross-sectional shape of round, oval or polygonal, or a combination thereof.

17. A method according to 10, wherein each of the rods has a diameter of 70 μm-5.0 mm.

18. A biomaterial comprising the material according to 1.

19. A drug controlled release body comprising the material according to 1.

20. A tissue engineering scaffold comprising the material according to 1.

21. A material according to 1, comprising a plurality of coplanar penetration pores.

22. A material according to 20, comprising a second plurality of coplanar penetration pores that are disposed in a different plane than the first plurality of coplanar

23. A material according to 22, wherein the second plurality of coplanar penetration pores are disposed in different directions than the first plurality of coplanar penetration pores.

24. A material according to 23, wherein the second plurality of coplanar penetration pores are substantially perpendicular to the first plurality of coplanar penetration pores.

### BRIEF DESCRIPTION OF THE DRAWINGS

FIG. 1 is a schematic view of a cured porous calcium phosphate material having complete communication pores and containing collagen, having a size of 10 mm×10 mm×8 mm.

FIG. 2 is a graph showing changes in weight of the cured materials after they are inplanted into the muscles of the rats.

FIG. 3 is a schematic view of a cured porous calcium phosphate material having complete communication pores and containing collagen after having been implanted in the muscles of a rat for 56 days.

FIG. 4 is a schematic view of a cured porous calcium phosphate material having no complete communication pores and containing no collagen after having been implanted in the muscles of a rat for 56 days.

FIG. 5 is a graph showing the effect of the number of communication pores on elution of indomethacin from the cured materials.

FIGS. 6-1, 6-2, and 6-3 are photos of the low-temperature curable calcium phosphate porous cured materials containing different collagen amounts. FIG. 6-1 is a photo of the cured material containing 20 mass percent of collagen. FIG. 6-2 is a photo of the cured material containing 30 mass percent of collagen. FIG. 6-3 is a photo of the cured material containing 40 mass percent of collagen.

FIGS. 7-1, 7-2, and 7-3 are photos of the low-temperature curable calcium phosphate porous cured materials containing collagen and various drugs. FIG. 7-1 is a photo of the cured material containing 20 mass percent of collagen+3 mass percent of cephalexin. FIG. 7-2 is a photo of the cured material containing 20 mass percent of collagen+3 mass percent of cephalexin+3 mass percent of indomethacin. FIG. 7-3 is a photo of the cured material containing 20 mass percent of collagen+3 mass percent of cephalexin+3 mass percent of menatetrenone.

FIG. 8 shows photos of the low-temperature curable calcium phosphate porous cured materials containing collagen cured at 80 Celsius degree.

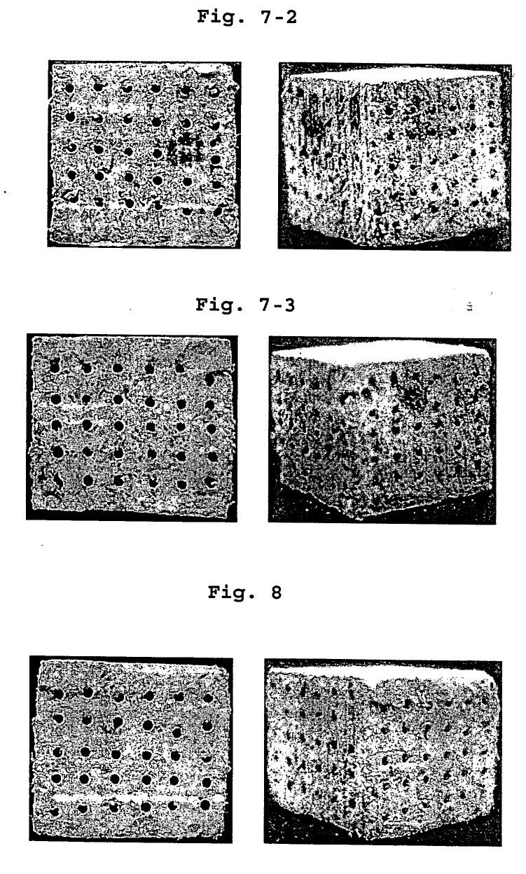

### Detailed Description of the Invention

As used herein “curing” means solidification defined by a final curing time in accordance with JIST6602. Preferably, a cured material of the invention is solidified such that a needle mark does not remain on the surface. An example of such a needle has a mass of 453.6 g and a cross-sectional area of 1.06 mm.

As used herein “deliberately-formed, three-dimensional penetration pores” herein means penetration pores deliberately, as opposed to randomly, formed in certain orientations and positions using longitudinal rods or similar forms or molds. Preferably, there are two or more penetration directions. Preferably, penetration start and end points are deliberately designed to optimize functionality of the material of the invention. The pores preferably fully penetrate the cured material of the invention. Preferably, spaces between the pores and positions of the pores are deliberately, as opposed to randomly, formed.

According to the present invention, the deliberately-formed, three-dimensional penetration pores are produced using a number of longitudinal rods. In one embodiment, each rod has a cross-sectional width of 90 μm to 5.0 mm, preferably 100 μm to 3.0 mm, and a length at least 3 times the cross-sectional width, preferably at least 10 times the cross-sectional width. Any material can be used for the rods as long as it does not readily react with the liquid component or the drug. Examples include stainless steel, wood, bamboo, other plant materials, carbon materials, polyethylene, nylon, polyacetal, polycarbonate, polypropylene, polyester, ABS, polystyrene, phenol, urea resin, epoxy resin, acrylite and the like.

The rods may have any cross-sectional shape. Press forming or drawing of the rod can be advantageously performed when the rod has one of the following cross-sectional shapes: a polygon having at least one set of parallel sides, an oval, a circle, a shape having at least one set of parallel sides, and a curved line. Preferably, the rods are straight, curved in a single plane, or in the shape of a polygonal line in the longitudinal direction. However, if the rods are curved in two or more planes, they may be deformed upon press forming and broken, or the molded material may be broken upon extraction of such rods after pressing.

The cross-sectional width of the rod is determined by the size of the pore that is finally needed. In the case of the calcium phosphate porous cured material for artificial bone or tissue engineering scaffold, the cured porous material preferably has pores into which a plurality of blood vessel endothelial cells or osteoblasts having an approximate size of 30 μm can penetrate simultaneously. In the case of the calcium phosphate porous cured material for artificial bone or tissue engineering scaffold, blood vessels with a diameter of 4 mm or more do not necessarily penetrate into the pores. Therefore the rod does not normally have a cross-sectional width of over 5.0 mm. In view of the above, the rod preferably has a cross-sectional width of 70 μm to 5.0 mm. The rod preferably has a length of at least 3 times the cross-sectional width, more preferably at least 10 times the cross-sectional width. If the length of the rod has a length less than 3 times the cross-sectional width, the maximum size of the final cured porous material, wherein all of the rods penetrate the powder, is limited to about 10 mm.

The rods are disposed in a desired position, and then a calcium phosphate cured material precursor mixed with a liquid component is added thereto. The rods may be disposed parallel to one another at equal spaces or at different spaces or non-parallel. Preferably, the rods do not overlap. Alternately, the rods may be disposed radially so that they aim at one point or plural points, or they may be disposed in dendrite structure. In this case, end faces of the rods are preferably completely contacted with, adhered to, or fitted with other rods. Insufficient contact may result in pores formed with a blind alley structure after curing.

As used herein, the “cured calcium phosphate material precursor” means calcium phosphate that can be hydrated and cured into hydroxyapatite, low crystalline hydroxyapatite, or calcium-deficient apatite. The precursor may contain as a second component an optimal amount of hydroxyapatite, low crystalline hydroxyapatite, or calcium-deficient apatite. The optimal amount of the second component promotes hydration and curing of the calcium phosphate precursor. Examples of the cured calcium phosphate material precursor include high-temperature-type tri-calcium phosphate, low-temperature-type tri-calcium phosphate, tetra-calcium phosphate, hydrogen calcium phosphate, hydrogen calcium phosphate dihydrate, octa-calcium phosphate, amorphous calcium phosphate, or calcium phosphate obtained by mixing two or more of the foregoing such that a molar ratio of powder calcium : phosphorus is 0.1 to 5.0, preferably 0.5 to 2.5, more preferably 1.3 to 1.8. The range of the molar ratio is determined based on a ratio of calcium : phosphorus 1.67 in hydroxyapatite.

The liquid component mixed with the cured calcium phosphate material precursor may be water, but preferably contains water-soluble inorganic salts, inorganic acid, or organic acid.

Examples of reaction promoters include phosphoric acid, succinic acid, malic acid, acetic acid, pseudo body fluid, phosphate buffer, saline, Ringer solution and the like, which can be used alone or in combination.

The cured porous calcium phosphate material prepared from the mixture having successive communication pores absorbs carbonic acid in air and changes into carbonate-containing apatite, which has high living body affinity similar to living body bone.

After curing, the mixture may be heated at 100° C.-1200° C. additionally, preferably 200° C.-800° C., more preferably 300° C.-500° C., whereby the cured porous calcium phosphate material may be sintered or cured to have great strength. Thus, the heated mixture is changed into apatite having complete communication pores with living body affinity, and then sintered or cured. At a low temperature of 100° C. or less, the mixture is not readily solidified. At a high temperature of 1200° C. or more, the hydroxyapatite produced is decomposed. At about 400° C., carbonic acid of carboxyapatite contained in the cured material remains and is sintered as carboxyapatite to provide both high living body affinity and mechanical strength.

According to the present invention, the cured porous calcium phosphate material increases in strength when the cured porous calcium phosphate materials is heated.

The cured calcium phosphate material precursor may be mixed with a natural or synthetic polymer having biocompatibility such as collagen, gelatin, chitin, chitosan, agarose, hydroxypropyl cellulose, hydroxypropyl methylcellulose, hydroxypropyl methylcellulose phthalate, hydroxypropyl methylcellulose acetate succinate, methylcellulose, ethylcellulose, hydroxyethyl cellulose, pullulan, aminoalkyl methacrylate copolymer, methacrylic acid copolymer, carboxyvinyl polymer, polyvinyl alcohol, dimethyl polysiloxane, sodium alginate, alginic acid pyrene glycol ester, polyvinyl pyrrolidone, agar and the like. The precursor can contain 0.1 to 90% by weight, preferably 5 to 50% by weight, and more preferably 10 to 30% by weight of the polymer. If the amount of polymer is less than 0.1%, the polymer's biocompatibility affinity is likely to not be apparent. If the amount of polymer exceeds 90%, the amount of inorganic components will likely be insufficient. If the amount of polymer exceeds 90%, the cured material precursor is not set even after transformation of the precursor into apatite. As a result, the fracture strength of the cured material becomes extremely low.

By adding the polymer, a cured material having great mechanical strength can be prepared without sintering at high temperature. The biocompatible polymer further enhances the biocompatibility of the cured material.

The cured calcium phosphate material has high biocompatibility and preferably has complete communication pores to effectively receive osteoblasts and osteoclasts. In order to supply nutrients for aiding their activity, new blood vessels are formed. Remodeling of the bones is thus accelerated. In other words, osteoblasts form bones in areas where the bones are expected to be newly formed, and osteoclasts induce biodegradation rapidly in areas where the bones are not formed. These activation properties are effectively induced by the drug included to provide the artificial bones with high bone-forming ability, and long-term, i.e., year-to-year, controlled drug release based on biodegradation and absorption.

A single-layer, pressed molded product is prepared using the mixture of the calcium phosphate precursor and the liquid component, if used, and the drug. A plurality of molded products are then laminated. A plurality of single-layer molded products prepared in advance may be laminated at one time. Alternatively, after a single layer is pressed and molded, another single layer may be formed and laminated thereon. In the lamination step, the rods in one layer are preferably contacted with the rods above and below at multiple contact points. The directions of the rods are preferably different in the adjacent layers. The contact points of the rods become successive pores in the lamination direction. The pressure for pressing and forming is 0.0001 MPa to 10 Mpa, and preferably 0.001 MPa to 1 MPa. If the pressure is less than 0.0001 MPa, pressure is generally not well applied. If the pressure exceeds 10 MPa, the rods may be deformed or broken depending on the material.

The mixture of the calcium phosphate precursor and the liquid component can contain a drug for improving bone formation or other living body functions. The drug may be mixed with the calcium phosphate precursor powder, may be dissolved or suspended in the liquid component in advance, or may be added when the calcium phosphate precursor and the liquid component are mixed. Examples of the drug include an antirheumatic agent such as di-sodium lobenzarit, bucillamine, Acralite salazosulfapyridine, farnesyl acid predonisone; an immunosuppression agent such as methotrexate, an arthrifuge such as colchicine, sulfan pyrazone, probenecid bucolome, benzbromarone, allopurinol; an antidiabetic agent such as insulin, isoinsulin, protamine zinc isgyline, glibenclamide, tolbutamide, acetohexamide, tolazamide, glybuzole and troglitazone; a sex hormone agent such as estradiol, ethinylestradiol, estriol, mestranol, progesterone, chlormadinon acetate, and methyltestosterone; a hormone agent such as gonadorelin acetate, somatolerin acetate, tetracosactide acetate, vasopressin, glucagon and epitiostanol; a protein bone growth factor such as calcitonin, interleukin-1, interleukin-6, a bone growth factor, an insulin-like simulating factor and a fibroblast growth factor; a bone metabolic improver such as alpha calcidiol, menatetrenone, elcatonin, ipriflavone, di-sodium etidronate, sodium alendronate hydrate; a cardiac drug such as digoxin, aminophylline, dopamine hydrochloride and mihinone; an antiarrhythmic agent such as disopyramide phosphate and pimenol hydrochloride; an antibacterial agent such as cephalexin, cephalothin sodium, gentamicin antibiotic, nitrofurantoin and fosfomycin sodium; an carcinostatic such as cytarabine, mercaptopurine, fluorourasil, 6-mercaptopurine, tegafur and methotrexate; and an anti-inflammatory agent such as indomethacin.

Different drugs may be used in combination. The roles of the drugs can be assigned for synergism and difference in release rate. For example, an antibiotic may be used to preventatively suppress bacterial contamination during surgical treatment. After the antibiotic plays its role, a bone-growing factor may be used to promote growth of the bones. The drug may be mixed with the calcium phosphate precursor or the liquid component in an amount of 1 ppb to 50 w/w %, preferably 1 ppm to 30%, and more preferably 100 ppm to 10%. The cured porous calcium phosphate material having complete communication pores is provided as discussed above. If the amount of the drug is less than 1 ppb, the drug may not be effective. If the amount of the drug exceeds 50 w/w %, the cured material may not be solidified.

According to the present invention, the cured porous calcium phosphate material is preferably produced by adding the liquid component to the calcium phosphate precursor powder, adding, if desired, the drug, pressing and forming the mixture, allowing the product to stand after the lamination step, and then curing the laminated product until the shape is held. The curing temperature is not especially limited, but may be 10° C. to 100° C., 25° C. or more at 80% or more humidity, and is preferably 25° C. or more to less than 80° C. at 80% or more humidity. This prevents the liquid component from evaporating until the crystal transition to the cured material is completed. The curing time is not especially limited, but is preferably 10 minutes to 8 hours. If the curing time exceeds 8 hours, bacteria may propagate, leading to increased amounts ofpyrogeneous substances. If the curing time is less than 10 minutes, the hardness may be insufficient.

After curing, the porosity can be determined by measuring the density. Communication of the pores can be evaluated using a microscope, a stereoscope, an electron microscope, or dye penetration. A specimen of the cured porous material with a diameter of approximately 6 mm-12 mm is set on the Instron type universal testing machine for evaluating its compressive strength. The drug controlled release properties of the porous cured material can be evaluated in accordance with Drug Release Rate Testing Method (Japan Pharmacopeia General Testing Method). For example, the cured material is agitated with a paddle at 100 rpm and at 37° C. in 900 mL of pH 7.4 pseudo body fluid or phosphate buffer. A portion of the solution is sampled for the drug release measurement by ultraviolet absorption, atomic absorption spectrophotometry, or high performance liquid chromatography.

The calcium phosphate porous cured material attained by the present invention is useful not only as a biomaterial, a tissue engineering scaffold and a drug support medium for DDS that require biological affinity, but also for any auxiliary member of an artificial heart or bone that is introduced into a body.

### EXAMPLE 1

The following three types of cured materials were produced:
(1) Porous cured material having complete communication pores and containing collagen
(2) Porous cured material having no complete communication pores and containing no collagen
(3) Porous cured material having no complete communication pores and containing collagen

Tetra-calcium phosphate (TTCP) and hydrogen calcium phosphate dihydrate (DCPD) were weighed at a molar ratio of 1:1, and were mixed and milled using a vibrating mill for 10 minutes. As to each of the collagen-containing porous cured materials, Type I collagen was mixed with the above-described mixture at a weight percent of 25%, and milled using a vibrating mill for 20 minutes. (TTCP: 408.23 mg, DCPD: 191.76 mg, collagen: 150.00 mg). 750 mg of the mixture was mixed with 600 μL of an 11 mMol phosphate solution. The complete communication pores were produced as follows: Six stainless steel rods having a diameter of 0.5 mm and a length of 28 mm were disposed parallel at a spacing of 1.54 mm. Another six stainless steel rods having the same size were disposed thereon in the transverse direction. The spaces between the rods were filled with the mixture, packed, and pressed at 0.01 MPa. Five layers were laminated. The mixture was allowed to stand and self-cure at 37° C. and 100% relative humidity for 24 hours.

To form the porous materials having complete communication pores, the rods were removed. Each cured material was removed from its mold, allowed to stand under reduced pressure for 24 hours, and dried to provide a cured material sample having a length of 10 mm, a wide of 10 mm and a height of 8 mm.

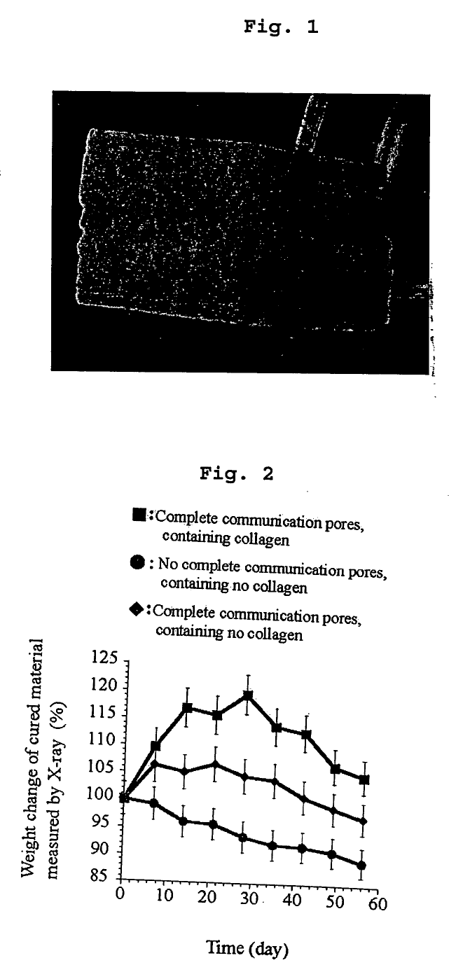

To form (1) porous cured material having complete communication pores and containing collagen, straight line penetration pores each having a diameter of 500 μm were disposed at a spacing of 1540 μm, and some rows of the pores were crossed alternately (FIG. 1). In this case, intersection points of the pores in the two directions were closed. Porosity was 23% on average. Sufficient mechanical strength was obtained.

### EXAMPLE 2

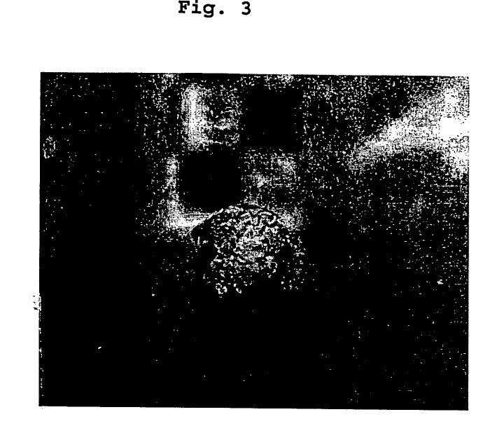

The three types of the cured materials in Example 1 were implanted buried into back muscles of rats. FIG. 2 shows weight changes of the cured materials measured by X-ray transmission for 56 days after the cured materials were implanted in the back muscles of the rats. The density of the porous cured material having complete communication pores and containing collagen increased for 30 days, and then decreased.

Density of the porous cured material having no complete communication pores and containing no collagen slightly increased after implantation, and then decreased gradually. This indicates that the cured material was biodegraded and eroded.

Density of the porous cured material having no complete communication pores and containing collagen slightly decreased after implantation. This indicates that the cured material was dissolved. However, there was no external erosion affecting the shape of the cured material after 56 days.

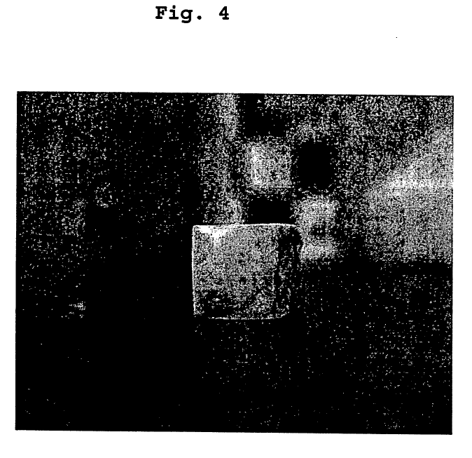

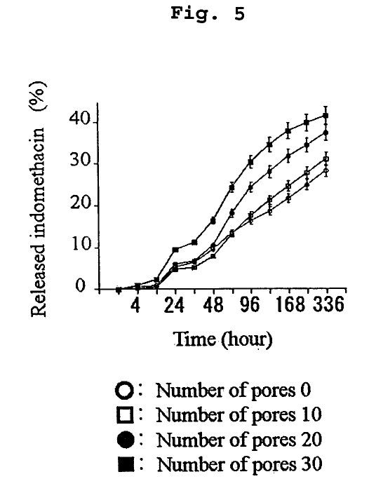

Thus, the density of the porous cured material having the complete communication pores and containing collagen increased temporarily and then decreased, and the porous cured material having no complete communication pores and containing collagen had different living body activity. FIGS. 3 and 4 show photographs of the cured materials after having been implanted in the back muscles of the rats for 56 days. After 56 days, the porous cured material having complete communication pores and containing collagen was biodegraded and eroded (FIG. 3). On the other hand, the porous cured material having no complete communication pores and containing no collagen was biodegraded but less eroded (FIG. 4). In view of the above, depending on the presence or absence of complete communication pores and collagen, the manner and the rate of biodegradation are significantly different.

### EXAMPLE 3

Calcium phosphate cured material containing an in vitro drug, having 10 complete communication pores, containing collagen.

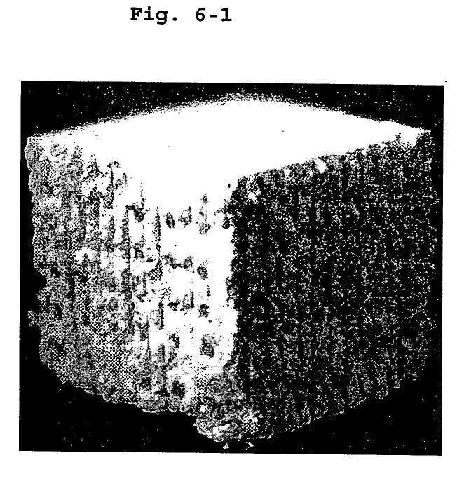

Tetra-calcium phosphate (TTCP) and hydrogen calcium phosphate dihydrate (DCPD) were weighed at a molar ratio of 1:1, and were mixed and milled using a vibrating mill for 10 minutes. Type I collagen was mixed with the above-described mixture at a weight percent of 25%, and milled using a vibrating mill for 20 minutes. Indomethacin (IMC) was weighed so that it occupied 3% of cement weight. (TTCP: 400.58 mg, DCPD: 188.17 mg, collagen: 138.75 mg, IMC 22.5 mg). 750 mg of the mixture was mixed with 600 μL of the 11 mMol phosphate solution. The mixture was introduced into molds. The porous cured materials were produced by crossing alternately 0×0, 5×2, 5×4 and 5×6 layers so that the numbers of complete communication pores were 0, 10, 20 and 30. The storage conditions were the same as those of the cured materials in Example 1. Respective average porosities were 0, 8, 15, and 23%. The average diameter of the pores was 500 μm. These cured materials were immersed in pseudo body fluid to release IMC controllably. FIG. 5 shows the effects of the communication pores on the IMC release profiles from the cured materials. The greater the number of communication pores in the cured materials, the faster the drug, IMC, was eluted. The release of the drug indicates a zero order release profile. Even at 14 days after the elution, drug release continued. Long-term, i.e., several months, controlled drug release is expected.

### EXAMPLE 4

The following three types of cured materials containing different collagen amounts were produced:
(1) Porous cured material having complete communication pores and containing 20% of collagen
(2) Porous cured material having complete communication pores and containing 30% of collagen
(3) Porous cured material having complete communication pores and containing 40% of collagen

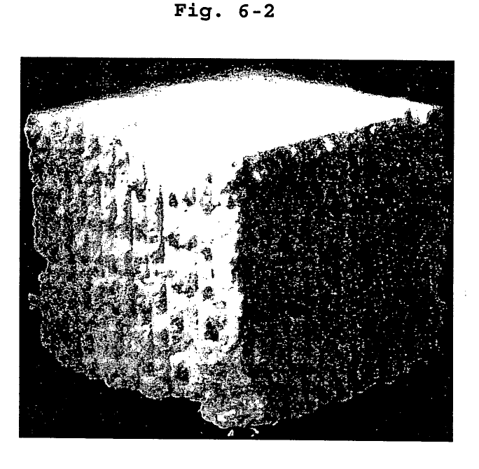

Tetra-calcium phosphate (TTCP) and hydrogen calcium phosphate dihydrate (DCPD) were weighed at a molar ratio of 1:1, and were mixed and milled using a vibrating mill for 10 minutes. To form the collagen-containing materials, Type I collagen was mixed with the above-described mixture at weight percents of 20-40%, and milled using a vibrating mill for 20 minutes. 750 mg of the mixture was mixed with 600 μL of the 11 mMol phosphate solution. The complete communication pores were produced as in Example 1. In the porous cured materials having the complete communication pores and containing collagen, straight line penetration pores each having a diameter of 500 μm were disposed at a spacing of 1540 Elm, and rows of the pores were crossed alternately (FIG. 6). Sufficient mechanical strength was obtained.

### EXAMPLE 5

The following cured materials containing drugs that play different roles were produced:
(1) Porous cured material having complete communication pores and containing 3% cephalexin and 20% collagen
(2) Porous cured material having no complete communication pores and containing 3% cephalexin, 3% indomethacin, and 20% collagen
(3) Porous cured material having no complete communication pores and containing 3% cephalexin, 3% menatetrenone, and 20% of collagen

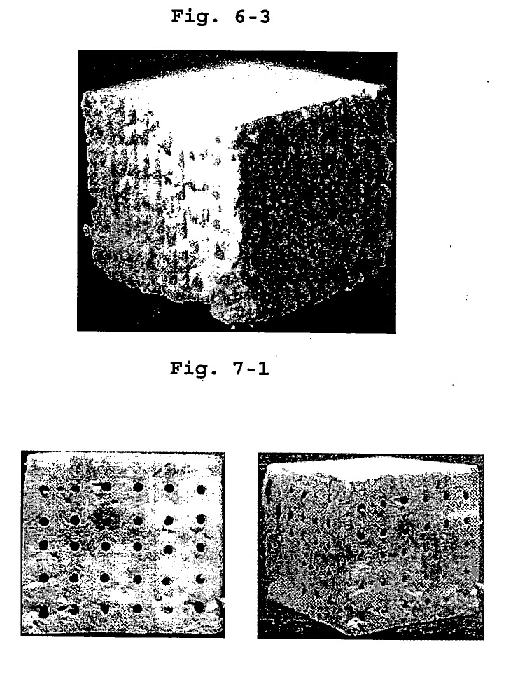

Tetra-calcium phosphate (TTCP) and hydrogen calcium phosphate dihydrate (DCPD) were weighed at a molar ratio of 1:1, and were mixed and milled using a vibrating mill for 10 minutes. To form the collagen-containing materials, Type I collagen was mixed with the above-described mixture at weight percents of 20%, and milled using a vibrating mill for 20 minutes. 750 mg of the mixture was mixed with 3% cephalexin, an antibiotic, and 3% an anti-inflammatory agent, a bone metabolic improver, menatetrenone (vitamin K2). Then, 600 μL of the 11 mMol phosphate solution was mixed therewith. The complete communication pores were produced as in Example 1. In the porous cured materials having the complete communication pores and containing collagen, straight line penetration pores each having a diameter of 500 μm were disposed at a spacing of 1540 μm, and rows of the pores were crossed alternately (FIG. 7). Sufficient mechanical strength was obtained.

### EXAMPLE 6

The following cured material was produced to investigate an effect of preparation temperature:

Cured porous material having no complete communication pores and containing 20% collagen prepared at 80° C.

Tetra-calcium phosphate (TTCP) and hydrogen calcium phosphate dihydrate (DCPD) were weighed at a molar ratio of 1:1, and were mixed and milled using a vibrating mill for 10 minutes. To form the collagen-containing body, Type I collagen was mixed with the above-described mixture at a weight percent of 20%. The complete communication pores were produced as in Example 1. The mixture was allowed to stand and self-cure at 80° C. and 100% relative humidity for 24 hours. Sufficient mechanical strength was obtained.

### EXAMPLE 7

The following cured material was produced to investigate the effects of preparation temperature and numbers of complete communication pores:

Porous cured material having complete communication pores and containing 20% collagen prepared at 80° C.

Tetra-calcium phosphate (TTCP) and hydrogen calcium phosphate dihydrate (DCPD) were weighed at a molar ratio of 1:1, and were mixed and milled using a vibrating mill for 10 minutes. To form the collagen-containing body, Type I collagen was mixed with the above-described mixture at a weight percent of 20%. The complete communication pores were produced as in Example 1. The mixture was allowed to stand and self-cure at 80° C. and 100% relative humidity for 24 hours. The complete communication pores were produced as follows: Six stainless steel rods having a diameter of 0.5 mm and a length of 28 mm were disposed parallel to one another at a spacing of 1.54 mm. Another six stainless steel rods having the same size were disposed thereon in the transverse direction. The spaces between the rods were filled with the mixture, packed, and pressed at 0.01 MPa. Five layers were laminated to provide 30 pores, and seven layers were laminated to provide 42 pores. Sufficient mechanical strength was obtained. It is be concluded that the low-temperature curing type calcium phosphate porous cured material containing collagen can be cured at 25° C. or more.

### EXAMPLE 8

The following types of cured materials were produced:

Porous cured material having complete communication pores and containing a cement of TTCP and DCPD at a molar ratio of 1.2:0.8

Porous cured material having complete communication pores and containing a cement of TTCP and DCPD at a molar ratio of 1.2:0.8 and 20% collagen

Tetra-calcium phosphate (TTCP) and hydrogen calcium phosphate dihydrate (DCPD) were weighed at a molar ratio of 1:1, and were mixed and milled using a vibrating mill for 10 minutes. To form the collagen-containing body, Type I collagen was mixed with the above-described mixture at a weight percent of 20%, and milled using a vibrating mill for 20 minutes. (TTCP: 490 mg, DCPD: 153 mg, collagen: 160 mg). 750 mg of the mixture was mixed with 600 μL of the 11 mMol phosphate solution. Similar procedures were repeated as in the previous Examples. Sufficient mechanical strength was obtained.

### EXAMPLE 9

The cured materials containing collagen and containing no collagen were produced as follows: Tetra-calcium phosphate (TTCP) and hydrogen calcium phosphate dihydrate (DCPD) were weighed at a molar ratio of 1:1, and were mixed and milled using a vibrating mill for 10 minutes. To form the collagen-containing cured material, Type I collagen was mixed with the above-described mixture at a weight percent of 20%, and milled using a vibrating mill for 20 minutes. 750 mg of the mixture was mixed with 600 μL of the 11 mMol phosphate solution. The mixture was allowed to stand and self-cure at 37° C. and 100% relative humidity for 24 hours. A portion of the cured material containing no collagen was heated in air by an electric furnace at 400° C. for 24 hours. All cured materials (diameter 6 mm×12 mm) were measured using the Instron type universal testing machine at a cross head speed of 10 nun/min. Results from the measurements are shown below.

TABLE 1
Compressive strength of calcium phosphate cured materials

| | |
|---|---|
| Cured material type | Compressive strength |

| | |
|---|---|
| containing no collagen + not heated | 85 kg/cm² |
| containing collagen + not heated | 205 kg/cm² |
| containing no collagen + heated | 380 kg/cm² |

These results show that the strength is improved by adding the optimal amount of a living body affinity polymer such as collagen to the calcium phosphate cured material, or heating the cured material after curing.

1. A cured porous calcium phosphate material, comprising at least one penetration pore formed therein, wherein said pore has a diameter of 70 μm to 4 mm, and wherein said material has a porosity of 20% to 80%.

2. A material according to claim 1, comprising a plurality of penetration pores arranged in a three-dimensional network structure.

3. A material according to claim 1, further comprising a biocompatible polymer.

4. A material according to claim 3, wherein the biocompatible polymer is at least one organic polymer selected from the group consisting of: collagen, gelatin, chitin, chitosan and hydroxypropyl methylcellulose.

5. A material according to claim 1, further comprising a drug.

6. A material according to claim 5, wherein the drug is at least one drug selected from the group consisting of: an antirheumatic agent; an immunosuppression agent; a sex hormone agent; a hormone agent; a protein bone growth factor; a bone metabolic improver; a cardiac drug; an antiarrhythmic agent; an antibacterial agent; an carcinostatic; and an anti-inflammatory agent.

7. A material according to claim 6, wherein the drug is an antirheumatic agent selected from the group consisting of di-sodium lobenzarit, bucillamine, Acralite salazosulfapyridine, and farnesyl acid predonisone.

8. A material according to claim 6, wherein the drug is an immunosuppression agent selected from the group consisting of methotrexate, an arthrifuge such as colchicine, sulfan pyrazone, probenecid bucolome, benzbromarone, and allopurinol.

9. A material according to claim 6, wherein the drug is an antidiabetic agent selected from the group consisting of insulin, isoinsulin, protamine zinc isgyline, glibenclamide, tolbutamide, acetohexamide, tolazamide, glybuzole and troglitazone.

10. A material according to claim 6, wherein the drug is a sex hormone agent selected from the group consisting of estradiol, ethinylestradiol, estriol, mestranol, progesterone, chlormadinon acetate, and methyltestosterone.

11. A material according to claim 6, wherein the drug is a hormone agent selected from the group consisting of gonadorelin acetate, somatolerin acetate, tetracosactide acetate, vasopressin, glucagon and epitiostanol.

12. A material according to claim 6, wherein the drug is a protein bone growth factor selected from the group consisting of calcitonin, interleukin-1, interleukin-6, a bone growth factor, an insulin-like simulating factor and a fibroblast growth factor.

13. A material according to claim 6, wherein the drug is a bone metabolic improver selected from the group consisting of alpha calcidiol, menatetrenone, elcatonin, ipriflavone, di-sodium etidronate, sodium alendronate hydrate.

14. A material according to claim 6, wherein the drug is a cardiac drug selected from the group consisting of digoxin, aminophylline, dopamine hydrochloride and milrinone.

15. A material according to claim 6, wherein the drug is an antiarrhythmic agent selected from the group consisting of disopyramide phosphate and pimenol hydrochloride.

16. A material according to claim 6, wherein the drug is an antibacterial agent selected from the group consisting of cephalexin, cephalothin sodium, gentamicin antibiotic, nitrofurantoin and fosfomycin sodium.

17. A material according to claim 6, wherein the drug is a carcinostatic selected from the group consisting of cytarabine, mercaptopurine, fluorourasil, 6-mercaptopurine, tegafur and methotrexate.

18. A material according to claim 6, wherein the drug is an anti-inflammatory agent which is indomethacin.

19. A material according to claim 1, which is low-temperature curable.

20. A material according to claim 1, which is heated and cured at 100-1200 degrees Celsius.

21. A material according to claim 1, wherein the penetration pore has a cross-sectional shape that is round, oval, polygonal, or a combination thereof.

22. A method of producing a cured porous calcium phosphate material, comprising the steps of:
disposing one or more rods in a single plane,
introducing into the space adjacent said one or more rods (a) a composition comprising a calcium phosphate cured material precursor and a liquid component, or (b) a composition comprising a calcium phosphate cured material precursor, a biocompatible polymer and a liquid component, whereby the composition surrounds the one or more rods,
curing the composition, and
removing the rods.

23. A method according to claim 22, wherein a plurality of rods are disposed in a single plane.

24. A method according to claim 23, wherein a plurality of second rods are disposed in a single plane in one or more directions on the first rods.

25. A method according to claim 24, wherein the second rods are disposed in different directions than the first rods.

26. A method according to claim 22, wherein the composition comprises a drug.

27. A method according to claim 22, wherein the volume percentage of the one or more rods is 5% to 90% of the cured material.

28. A method according to claim 22, wherein the one or more rods have a cross-sectional shape of round, oval or polygonal, or a combination thereof.

29. A method according to claim 22, wherein each of the rods has a diameter of 70 μm-5.0 mm.

30. A biomaterial comprising the material according to claim 1.

31. A drug controlled release body comprising the material according to claim 1.

32. A tissue engineering scaffold comprising the material according to claim 1.

33. A material according to claim 1, comprising a plurality of coplanar penetration pores.

34. A material according to claim 33, comprising a second plurality of coplanar penetration pores that are disposed in a different plane than the first plurality of coplanar penetration pores.

35. A material according to claim 34, wherein the second plurality of coplanar penetration pores are disposed in different directions than the first plurality of coplanar penetration pores.

36. A material according to claim 35, wherein the second plurality of coplanar penetration pores are substantially perpendicular to the first plurality of coplanar penetration pores.
